(19) United States
(12) Patent Application Publication
(10) Pub. No.: US 2026/0269014 A1
(43) Pub. Date: Sep. 10, 2026

(54) K-MER-BASED METHODS FOR ASSEMBLING POLYNUCLEOTIDE SEQUENCES

(71) Applicant: Illumina, Inc., San Diego, CA (US)

(72) Inventors: Aaron L. Halpern, San Diego, CA (US); Pierre Maxime Kevin Morisse, Paris (FR); Varun Jain, San Diego, CA (US); Michael Ruehle, San Diego, CA (US); Jeffrey Yuan, San Diego, CA (US); Aaron Earl Darling, Hornsby (AU)

(21) Appl. No.: 19/162,873

(22) PCT Filed: Mar. 7, 2024
(86) PCT No.: PCT/US2024/018815

§ 371 Date: Sep. 5, 2025

Related U.S. Application Data
(60) Provisional application No. 63/489,606, filed on Mar. 10, 2023.

(51) Int. Cl. G16B 30/20; G16B 30/00

(52) U.S. Cl. CPC G16B 30/20; G16B 30/00

(57) ABSTRACT

A method and system for polynucleotide sequencing and sequence assembly using a k-mer graph approach is disclosed. The methods may include receiving a sample comprising a nucleic acid sequence, fragmenting the sample into a plurality of shorter sequences, and aligning the shorter sequences to a reference genome to identify a k-mer sequence. The systems may include a processor, a memory, and a storage device configured to store the reference genome and the sample. The processor may be configured to execute the steps of the method, including aligning shorter sequences to a reference genome.

### CROSS-REFERENCE TO RELATED APPLICATIONS

This application claims priority to U.S. Provisional Application No. 63/489,606, filed Mar. 10, 2023, the content of which is incorporated by reference in its entirety.

### FIELD OF THE INVENTION

This disclosure relates to DNA sequencing and sequence assembly. More particularly, this disclosure relates to nucleotide sequence assembly methods by assembling numerous small sequences using a k-mer approach.

### BACKGROUND

Identifying disease-causing mutations in DNA has been a long-term goal of genetic medicine. Genomic variants can be either small changes to patients' DNA sequences or larger deletions and duplications. Existing sequencing technology is comparatively adept at detecting single-nucleotide polymorphisms (“SNPs”) and short insertions/deletions (“indels”) because these individual mutations retain the context of the nearby sequence. Some diseases are caused by more extensive mutations, like alpha-thalassemia caused by the whole-gene deletions of HBA1 and HBA2. Next-generation sequencing “NGS,” developed over the last decade, is increasingly capable of confidently identifying the differences between a patient's and a reference genome. NGS refers to a diverse collection of post-Sanger sequencing technologies, including sequencing-by-synthesis, sequencing-by-ligation, ion semiconductor sequencing, etc.

NGS has been the most widely adopted technology for human whole-genome sequencing (WGS), delivering an accurate, scalable, cost-effective solution featured in over 300,000 scientific publications. However, a small portion of the genome remains challenging to map due to highly repetitive or homologous regions. Standard short-read sequencing provides accurate base-level sequences to provide short-range information but may underperform in providing long-range information. NGS technology and assembly methods aim to address such edge cases and to accelerate access to the remaining ~5% of genetic regions that are currently challenging to map.

Additionally, while NGS methods are widely used for large-scale DNA sequencing projects, such methods can be expensive and time-consuming. An alternative assembly method may consider short substrings of fixed length k, known as k-mers. A k-mer approach to assembly involves fragmenting a sample into a plurality of shorter sequences and aligning the shorter sequences to a reference genome to identify a progressively longer k-mer series.

### SUMMARY

An aspect of the disclosure is directed to sequencing methods and systems that produce high-quality assemblies. For example, some embodiments may produce acyclic k-mer graphs. Some embodiments apply a globally optimal procedure for determining the heaviest path of edge weights on acyclic k-mer graphs. Such embodiments may improve upon traditional methods that rely on greedy path extraction for assembling k-mer graphs or even on greedy path extraction with lookback because greedy procedures may fail to find the globally optimal solution because the procedures focus on local information and not all the available data. The choice made by a greedy procedure for one path on a k-mer graph may depend on choices the greedy procedure already made upstream, which can compound errors and lead to lower confidence in an assembly. In one aspect, a globally optimal procedure may be applied where a k-mer graph cannot be optimally assembled by using optimal solutions of its subproblems. The methods and systems of the disclosure may improve upon greedy methods by finding, within each subset of unaligned reads to assemble, the globally best assembly or assemblies in terms of explaining the highest fraction of the input reads.

In some embodiments, the k-mers of the reference guides may be oriented relative to a representative reference sequence segment. Embodiments may also include generating, using the k-mers of the reference guides and of the reads, an oriented k-mer graph. In some embodiments, the k-mers of the reads of genomic data may be sequentially added to the k-mer graph. Embodiments may also include determining the polynucleotide sequence using the oriented k-mer graph.

Embodiments of the present disclosure may utilize mutated read sequences, and such methods and systems may include generating aligned mutated read sequences and genetic reference sequence segments. Embodiments of the present disclosure may also use a cluster file and any associated data. In some embodiments, the cluster file may include read sequences and an assignment of the read sequences to a cluster. In some embodiments, the cluster file may include greater than 1 million read sequences. In some embodiments, a generated aligned sequence may include greater than 100 subgraphs. In some embodiments, the information on the orientation of at least some of the reads of genomic data may include information on the relative orientation of overlapping sequences of read pairs. In some embodiments, the length of the reads of genomic data ranges from 25 bp to 300 bp.

Embodiments of the present disclosure may also include methods for generating, using a BAM file and data structure, a reference guide with reference sequence segments as k-mers. In some embodiments, methods may include generating, using the k-mers of the reference guides and the reads, a k-mer graph. In some embodiments, methods may be directed to sequencing experiments that only use acyclic k-mer graphs. In addition, or in the alternative, in some embodiments, methods may be able to advantageously produce acyclic k-mer graphs at a significantly higher rate. A significantly higher rate of producing acyclic k-mer graphs may be a rate that does not require or provide a benefit from filtering sequences of acyclic k-mer graphs. In some embodiments, the method may include determining, using a globally optimal procedure, the heaviest path in a k-mer graph.

Some embodiments relate to a method of assembling the nucleotide sequence of a polynucleotide from read pairs, including: providing a flowcell including clusters of read pairs, wherein each read pair contains two sequence reads, and the two sequence reads are in opposite orientations of one another; mapping a plurality of the read pairs to a reference guide of the polynucleotide; orienting sequence reads in each read pair to be in the same orientation as the reference guide of the polynucleotide; generating, from the oriented sequence reads, at least one k-mer graph including nodes and edges, wherein each node of the at least one k-mer graph corresponds to the read pairs from a single cluster and each edge has a weight corresponding to the number of identical sequence reads in the cluster; and assembling the nucleotide sequence of the polynucleotide by tracing a plurality of acyclic paths through the at least one k-mer graph.

Another embodiment relates to a system for analyzing sequences of polynucleotides having a machine-readable memory and a processor configured to execute machine-readable instructions, which, when executed by the processor, cause the system to perform steps described above and herein.

Another embodiment is a non-transitory computer-readable medium storing a polynucleotide sequence analyzing program including instructions that, when executed by a processor, causes a polynucleotide sequencing apparatus to perform steps described above and herein.

### BRIEF DESCRIPTION OF THE DRAWING

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a flowchart illustrating a process of sequencing a reference genome by inputting polynucleotide cluster data into a k-mer assembly system and outputting assembled sequence data files.

FIG. 2 depicts an embodiment of a system including a memory comprising a cluster assembly process module.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

The following description and examples illustrate an embodiment of the present invention in detail. Those of skill in the art will recognize that there are numerous variations and modifications of this invention that are encompassed by its scope. Accordingly, the description of a preferred embodiment should not be deemed to limit the scope of the present invention.

### Overview

Embodiments relate to systems and methods for efficiently determining the sequence of relatively long nucleic acid molecules by fragmenting the nucleic acid molecules into relatively short fragments, sequencing those fragments to determine a sequencing read, and then assembling the sequencing reads to determine the final sequences of the long nucleic acid molecules. In general, a sequencing read (also referred to as a “read”) refers to the inferred sequence of nucleotide bases in a nucleic acid molecule. A sequencing read may be of any appropriate length, such as about or more than about 20 nt, 30 nt, 36 nt, 40 nt, 50 nt, 75 nt, 100 nt, 150 nt, 200 nt, 250 nt, 300 nt, 400 nt, 500 nt, or more in length. In some embodiments, a sequencing read is less than 200 nt, 150 nt, 100 nt, 75 nt, or fewer in length. Sequencing reads can be “paired,” meaning that they are derived from different ends of a nucleic acid fragment. Paired reads can have intervening unknown sequence or overlaps with one another. In some cases, the sequencing read is a contig or consensus sequence assembled from separate overlapping reads. A sequencing read may be analyzed in terms of component k-mers. In general, “k-mer” refers to the subsequences of a given length k that make up a sequencing read. For example, the sequence “AGCTCT” can be divided into the 3 nucleotide subsequences “AGC,” “GCT,” “CTC,” and “TCT.” In this example, each of these subsequences is a k-mer, wherein k═3. K-mers may be overlapping or non-overlapping.

Sequence comparison may comprise one or more comparison steps in which one or more k-mers of a sequencing read are compared to k-mers of one or more reference sequences (also referred to simply as a “reference”). In some embodiments, a k-mer is about or more than about 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 75 nt, 100 nt, or more in length. In some embodiments, a k-mer is about or less than about 30 nt, 25 nt, 20 nt, 15 nt, 10 nt, or fewer in length. The k-mer may be in the range of 3 nt to 13 nt, 5 nt to 25 nt in length, 7 nt to 99 nt, or 3 nt to 99 nt in length. The length of k-mers analyzed at each step may vary. For example, a first comparison may compare k-mers in a sequencing read and a reference sequence that are 21 nt in length, whereas a second comparison may compare k-mers in a sequencing read and a reference sequence that are 7 nt in length. For any given sequence, in a comparison step, the k-mers being analyzed may be overlapping (such as in a sliding window) and may be of same or different lengths. While k-mers are generally referred to herein as being made from nucleic acid sequences, it should be realized that a k-mer may also encompass polypeptide sequences, including comparison of k-mers consisting of amino acids.

A reference sequence may include any sequence to which a sequencing read is compared. Typically, the reference sequence is associated with some known characteristic, such as a condition of a sample source, a taxonomic group, a particular species, an expression profile, a particular gene, an associated phenotype such as likely disease progression, drug resistance or pathogenicity, increased or reduced predisposition to disease, or other characteristic. Typically, a reference sequence is only one of many such reference sequences in a sequence database. A variety of databases comprising various types of reference sequences are available, one or more of which may serve as a reference database either individually or in various combinations. Databases can comprise many species and sequence types, such as NR, UniProt, SwissProt, TrEMBL, or UniRef90. Databases can comprise specific kinds of sequences from multiple species, such as those used for taxonomic classification of species, such as bacteria.

Databases can comprise multiple sequences from a single species, such as the human genome, the human transcriptome, model organisms such as the mouse genome, the yeast transcriptome, or the C. elegans proteome, or disease vectors such as bat, tick, or mosquitoes and other domestic and wild animals. In some embodiments, the reference database comprises sequences of human transcripts. Reference sequences in databases can comprise DNA sequences, RNA sequences, or protein sequences. Reference sequences in databases can comprise sequences from a plurality of taxa. In some cases, the reference sequences are from a reference individual or a reference sample source. Examples of reference individual genomes are, for example, a maternal genome, a paternal genome, or the genome of a non-cancerous tissue sample.

Examples of reference individuals or sample sources are the human genome, the mouse genome, or the genomes of particular serovars, genovars, strains, variants or otherwise characterized types of bacteria, archea, viruses, phages, fungi, and parasites. The database can comprise polymorphic reference sequences that contain one or more mutations with respect to known polynucleotide sequences. Such polymorphic reference sequences can be different alleles found in the population, such as SNPs, indels, microdeletions, microexpansions, common rearrangements, genetic recombinations, or prophage insertion sites, and may contain information on their relative abundance compared to non-polymorphic sequences. Polymorphic reference sequences may also be artificially generated from the reference sequences stored in a database, such as by varying one or more (including all) positions in a reference genome such that a plurality of possible mutations not in the actual reference database are represented for comparison. The database of reference sequences can comprise reference sequences of one or more of a variety of different taxonomic groups, including but not limited to bacteria, archaea, chromalveolata, viruses, fungi, plants, fish, amphibians, reptiles, birds, mammals, and humans. In some cases, the database of reference sequences consists of sequences from one or more reference individuals or a reference sample sources (e.g. 10, 100, 1000, 10000, 100000, 1000000, or more), and each reference sequence in the database is associated with its corresponding individual or sample source. In some embodiments, an unknown sample may be identified as originating from an individual or sample source represented in the reference database on the basis of a sequence comparison.

In some embodiments, each reference sequence in the database of reference sequences is associated with, prior to the comparison, a k-mer weight as a measure of how likely it is that a particular k-mer within the reference sequence originates from the reference sequence. Calculating the k-mer weight can comprise comparing a reference sequence in the database to the other reference sequences in a database, such as by one or more methods described herein. The k-mer values thus associated with sequences in the database may then be used in determining the k-mer weights for k-mers within sequencing reads.

A collection of k-mers with or without assigned weights may be utilized in numerous tasks in bioinformatics analyses, many of which are configured to count k-mers as a pre-processing step. While k-mers may be used for pre-processing of data, such as in taxonomic classification systems, k-mers are also used as described herein for assembly and graph construction. In a typical de Bruijn graph construction, for example, graphs will have nodes and edges, where the nodes are oligonuclotide sequences, and the edges imply that two oligonucleotides are identical to each other and overlapping except for the first base of a first k-mer and the last base of the next (for a given k-mer, left and right (k−1)-mers in the graph, respectively. Accordingly, an edge implies that a k-mer at a particular node may be extended by a single base and produce another k-mer that is connected to another node in the graph. The amount of support for an edge of the k-mer graph can be used to provide guidance in graph assembly and in deciding what path to take through the many nodes of the k-mer graph to construct a particular K+1 length sequence.

Some embodiments provide for extraction of contigs with particular k-mer counts from a weighted de Bruijn graph. In some embodiments, the weight of an edge may be calculated by counting the reads which have an exact match to the sequence implied by the edge. As an example, if only one read has a particular edge k-mer, then that edge will have weight 1. If, however, five reads have that particular k-mer, then the edge will have weight 5, and so on. Accordingly, the edge of the graph with a highest number of read pairs containing an exact match to the k-mer corresponding to the edge will have a higher weight than another edge of the graph with a lower number of read pairs containing an exact match to the k-mer corresponding to the edge.

In some embodiments, the guide sequences that are copied from the reference genome are not counted in assigning the corresponding edge weights, so edge weights as described herein may originate from the actual sequence reads that go into the assembly process. In some embodiments, a node weight (k-mer count) can be associated with every k-mer from the graph. In some embodiments, a weighted graph may be constructed directly from raw input sequences, without pre-counting. In some embodiments, canonical graphs may contain each k-mer in both of its forms (forward and reverse complement). In some embodiments, a similar data structure may be modeled by storing only one k-mer, implicitly modeling the other.

In general, comparing k-mers in a read to a reference sequence comprises counting k-mer which match in the read and reference sequence. The stringency for identifying a match may vary. For example, a match may be an exact match, in which the nucleotide sequence of the k-mer from the read is identical to the nucleotide sequence of the k-mer from the reference. Alternatively, a match may be an incomplete match, where 1, 2, 3, 4, 5, 10, or more mismatches are permitted. In addition to counting matches, the weight of each k-mer can be calculated by counting how many times a particular k-mer appears within a particular reference sequence, how many times the particular k-mer appears among a group of sequences comprising the reference sequence, and how many times the particular k-mer appears among all reference sequences in the database of reference sequences. In one embodiment, the k-mer weight is calculated according to the following formula, which calculates the k-mer weight as a measure of how likely it is that a particular k-mer (Ki) originates from a reference sequence (refi) as follows:

$${KW 〚 ref 〛}_{i} i ( {K}_{i} ) = ( ( {C}_{r e f} ( {K}_{i} ) ) / 〈 {C}_{d b} ( {K}_{i} ) ) ) / ( {C}_{d b} ( {K}_{i} ) ) / ( Total k - mer count ) ) \qquad ( Eqn . 1 )$$

C represents a function that returns the count of Kᵢ. C_{ref}(Kᵢ) indicates the count of the Kᵢ in a particular reference. C_{db}(Kᵢ) indicates the count of Kᵢ in the database. This weight provides a relative, database specific measure of how likely it is that a k-mer originated from a particular reference. Prior to comparing a sequencing read to reference sequences, the k-mer weight (or measurement of likelihood that a k-mer originates from a given reference sequence) can be calculated for each k-mer and reference sequence in the database. In some examples, the k-mer weight of how likely it is that a k-mer originates from a specific taxon is calculated by defining C_{ref} (Kᵢ) in the above equation as a function that returns the total count of Kᵢ in a particular taxon.

One aspect of the disclosure is directed to introducing artificial mutations prior to sequencing that help sort reads into assembly clusters that need to be assembled. For the purposes of this description, a distinction is highlighted between a “sequencing cluster” and an “assembly cluster.” A “sequencing cluster” may refer to the set of DNA fragments created by, for example, bridge amplification of a single DNA fragment within a “spot” on a flow cell, which typically yields a single read pair. An “assembly cluster” may refer to a cluster of reads sharing mutations and inferred to be derived from the same mutated template. An “assembly cluster” may also generally refer to any cluster of reads inferred to be related. For example, some assembly clustering processes use single-linkage clustering, constructing a transitive closure of sequences with a similarity over a particular threshold.

In some embodiments, artificial mutations may provide an assembly cluster method to subset the assembly process down to a fraction of the overall polynucleotide assembly process. In some embodiments, the assembly clusters may individually correspond to single molecules of modest length relative to the whole genome. In some embodiments, artificial mutations may provide fingerprints to key certain short reads to corresponding assembly clusters. In some embodiments, artificial mutations may help to assemble short reads into longer reads/contigs, which can be mapped more readily to the reference genome. For example, two read pairs may share a sequence with some artificial mutations, which can enable a unique identification or grouping of the reads that belong to a single original molecule.

In some embodiments, an artificially mutated template is fragmented into smaller fragments (e.g. paired end inserts), which are sequenced. The resulting sequences may be decomposed into (overlapping) k-mers, where the length k is appreciably smaller than the length of the sequencing read. In some embodiment, the disclosed methods may use 150 bp sequencing reads, and use k=21, or 31 or 41 or 51 or 81. Other implementations for arriving at assembly clusters that need to be assembled are also contemplated.

As mentioned above, embodiments of the disclosure relate to systems and methods of determining the sequence of a target nucleic acid sequence by sequencing short segments of that target sequence and then assembling the short segments through a k-mer graphing process into the final target sequence. In some embodiments, the sequence reads are assembled by comparison to a reference genome. In some embodiments, the target sequence may be artificially mutated to contain mutant sequences (“mut” sequences) which help assemble short reads into longer reads/contigs, which can in turn be mapped to the reference genome more readily because the resulting reads are longer. In some embodiments, only the DNA is mutated. In some embodiments, fragments of the genome may be mutated prior to sequencing.

In such embodiments, a template sequence is randomly mutated and then fragmented into a plurality of shorter sequences of length “k”, referred to throughout the disclosure as “k-mers”. The nucleotide sequence of these k-mer sequences is then determined using, for example, next generation sequencing (“NGS”) techniques which are useful for rapidly and simultaneously sequencing a plurality of relatively short nucleotide sequences. In some embodiments, the NGS techniques first creates sequencing clusters of oligonucleotide sequences on a flow cell which are then read by a NGS systems, such as those manufactured by Illumina, Inc. (San Diego, CA). During the read process, each sequencing cluster may be used to read out the bound oligonucleotide sequences in the forward or reverse orientation, such that a pair of reads corresponding to the 5′-3′ and 3′-5′ orientation of the oligonucleotide being sequenced is determined. This read pair is then fed into the system, as described below, to generate a k-mer graph which may be used to assemble the sequenced oligonucleotide read pairs into the determined nucleotide sequence.

For example, a long target polynucleotide, such as a fragment of a genomic sequence, may be artificially mutated using a process as described in PCT Publication WO 2021/064365, herein incorporated by reference in its entirety. A mutated target polynucleotide is then fragmented, and each individual fragment is bound to a flowcell and amplified to create a sequencing cluster, but each sequencing cluster containing part of the mutated target polynucleotide might then be identified and said to correspond to an assembly cluster. The nucleotide sequence of the forward and reverse strand pairs within each assembly cluster can then be determined using NGS techniques.

An aspect of the disclosure is directed to creating a k-mer graph of all the k-mer sequences from the set of reads in an assembly cluster. By using a reference guide sequence, sequence reads can be initially assigned to a location along a putative reference guide polynucleotide. Each set of paired reads and reference guides may then be passed to a k-mer assembler system, which uses the paired reads and reference guides comprising potentially adjacent k-mer sequences to build a k-mer graph of the k-mer sequences from the sequencing run. Since each sequenced fragment may have a pair of reads, each in a different orientation, the system may determine which orientation in the read pair matches to the orientation of the reference guide sequence. From this information, the system may use the orientation information to provide all sequence data from a particular assembly cluster in the one orientation that is the same as the reference guide, as discussed in more detail below. It should be realized that multiple k-mer graphs may be built, with different k-mer sizes, if some k-mer sizes do not produce graphs with satisfactory predetermined features (e.g. graphs containing cycles or too many non-unique k-mers). Once a satisfactory k-mer graph is built for a particular k-mer size, a highest-scored path extraction procedure may be applied to assemble a long template from all of the k-mer graph data, which can then be output to a results file as described in more detail below.

One embodiment comprises determining the highest-scored path through the k-mer graph in order to assemble the target polynucleotide sequence from the k-mers. This can be accomplished by first finding the best forward path through the k-mer graph. Each k-mer graph contains nodes and edges. The nodes may contain the set of read pairs determined from each sequencing cluster that is sequenced on a flowcell. The edges are used to imply that two k-mers in two adjacent nodes are identical to each other except for the first base of one k-mer and the last base of the next k-mer. An edge implies the k-mer associated at a particular node has a sequence, and the adjacent node is that same k-mer sequence which has been extended by a single base to produce another k-mer corresponding to the adjacent node in the k-mer graph.

In one embodiment, the count of reads in an assembly cluster which have an exact match to the sequence implied by the edge may be calculated as the weight of that edge. Thus, an edge derived from an assembly cluster with a greater number of read pairs with identical sequences will have greater edge weight than an edge in the assembly cluster that has fewer identical read pairs. Once the k-mer assembly system calculates the set of non-cyclic paths forward from a first node to a final node, the system may calculate the highest-scored reverse path to determine the best fit for the assembled target sequence from each k-mer. By starting with a final node in each path, and then choosing to move backwards through the path by selecting nodes with the highest weight, the k-mer assembly system assembles a nucleotide sequence corresponding to the highest-scored path. In some embodiments, the disclosed methods and systems may employ dynamic programing methods, which may allow for determining the highest scored path without calculating the summed score for all possible paths. Dynamic programing methods described further herein allow for breaking the overall problem into segments, each of which may be simpler to calculate or otherwise solve.

In one aspect, the target sequence is a relatively long template read from an assay. Some embodiments of the disclosure enable the determination of contiguous long-read sequences with an N50 of 6-7 kb and read lengths of greater than 5 kb, 10 kb, 20 kb, 30 kb, 40 kb, 50 kb or more.

Certain embodiments of sequencing double-stranded polynucleotides using nanopores and/or certain embodiments of analyzing or organizing signals associated with k-mer polynucleotides can be utilized using systems or methods disclosed in U.S. provisional patent application Ser. No. 63/247,155 filed on Sep. 22, 2021 and U.S. patent application Ser. No. 17/224,496 published as US2021/0313009 on Oct. 7, 2021, the disclosures of which are incorporated herein by reference in their entireties. While some advantages of the disclosure are directed to short-read sequencing, assembly clustering and other methods of the disclosure are also contemplated as being applied to long-read sequencing.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

As used herein, the term “nucleotide” is intended to mean a molecule that includes a sugar and at least one phosphate group, and in some examples also includes a nucleobase. A nucleotide that lacks a nucleobase may be referred to as “abasic.” In some embodiments, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

Examples of nucleotides may include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

Examples of nucleotides may also be intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

In some embodiments, the term “modification” as used herein is intended to refer not only to a chemical modification of a nucleic acids, but also to a variation in nucleic acid conformation or composition, interaction of an agent with a nucleic acid (e.g., bound to the nucleic acid), and other perturbations associated with the nucleic acid. As such, a location or position of a modification is a locus (e.g., a single nucleotide or multiple contiguous or noncontiguous nucleotides) at which such modification occurs within the nucleic acid. For a double-stranded template, such a modification may occur in the strand complementary to a nascent strand synthesized by a polymerase processing the template, or may occur in the displaced strand. For example, modified nucleotides may include 5-methylcytosine, N6-methyladenosine, N3-methyladenosine, N7-methylguanosine, 5-hydroxymethylcytosine, pseudouridine, thiouridine, isoguanosine, isocytosine, dihydrouridine, queuosine, wyosine, inosine, triazole, diaminopurine, β-D-glucopyranosyloxymethyluracil (a.k.a., β-D-glucosyl-HOMedU, β-glucosyl-hydroxymethyluracil, “dJ,” or “base J”), 8-oxoguanosine, and 2′-O-methyl derivatives of adenosine, cytidine, guanosine, and uridine. Modified DNA and RNA bases are further described, for example, in Narayan P, et al. (1987) Mol Cell Biol 7 (4): 1572-5; Horowitz S, et al. (1984) Proc Natl Acad Sci U.S.A. 81 (18): 5667-71; “RNA's Outfits: The nucleic acid has dozens of chemical costumes,” (2009) C&EN; 87 (36): 65-68; Kriaucionis, et al. (2009) Science 324 (5929): 929-30; and Tahiliani, et al. (2009) Science 324 (5929): 930-35; Matray, et al. (1999) Nature 399 (6737): 704-8; Ooi, et al. (2008) Cell 133:1145-8; Petersson, et al. (2005) J Am Chem Soc. 127 (5): 1424-30; Johnson, et al. (2004) 32 (6): 1937-41; Kimoto, et al. (2007) Nucleic Acids Res. 35 (16): 5360-9; Ahle, et al. (2005) Nucleic Acids Res 33 (10): 3176; Krueger, et al., Curr Opinions in Chem Biology 2007, 11 (6): 588); Krueger, et al. (2009) Chemistry & Biology 16 (3): 242; Mccullough, et al. (1999) Annual Rev of Biochem 68:255; Liu, et al. (2003) Science 302 (5646): 868-71; Limbach, et al. (1994) Nucl. Acids Res. 22 (12): 2183-2196; Wyatt, et al. (1953) Biochem. J. 55:774-782; Josse, et al. (1962) J. Biol. Chem. 237:1968-1976; Lariviere, et al. (2004) J. Biol. Chem. 279:34715-34720; and in International Application Publication No. WO/2009/037473, the disclosures of which are incorporated herein by reference in their entireties.

Modifications may further include the presence of non-natural base pairs in the nucleic acid, including but not limited to hydroxypyridone and pyridopurine homo- and hetero-base pairs, pyridine-2,6-dicarboxylate and pyridine metallo-base pairs, pyridine-2,6-dicarboxamide and a pyridine metallo-base pairs, metal-mediated pyrimidine base pairs T-Hg(II)-T and C—Ag(I)—C, and metallo-homo-basepairs of 2,6-bis(ethylthiomethyl)pyridine nucleobases Spy, and alkyne-, enamine-, alcohol-, imidazole-, guanidine-, and pyridyl-substitutions to the purine or pyridimine base (Wettig, et al. (2003) J Inorg Biochem 94:94-99; Clever, et al. (2005) Angew Chem Int Ed 117:7370-7374; Schlegel, et al. (2009) Org Biomol Chem 7 (3): 476-82; Zimmerman, et al. (2004) Bioorg Chem 32 (1): 13-25; Yanagida, et al. (2007) Nucleic Acids Symp Ser (Oxf) 51:179-80; Zimmerman (2002) J Am Chem Soc 124 (46): 13684-5; Buncel, et al. (1985) Inorg Biochem 25:61-73; Ono, et al. (2004) Angew Chem 43:4300-4302; Lee, et al. (1993) Biochem Cell Biol 71:162-168; Loakes, et al. (2009), Chem Commun 4619-4631; and Seo, et al. (2009) J Am Chem Soc 131:3246-3252, the disclosures of which are incorporated herein by reference in their entireties). Other types of modifications include, e.g, a nick, a missing base (e.g., apurinic or apyridinic sites), a ribonucleoside (or modified ribonucleoside) within a deoxyribonucleoside-based nucleic acid, a deoxyribonucleoside (or modified deoxyribonucleoside) within a ribonucleoside-based nucleic acid, a pyrimidine dimer (e.g., thymine dimer or cyclobutane pyrimidine dimer), a cis-platin crosslinking, oxidation damage, hydrolysis damage, other methylated bases, bulky DNA or RNA base adducts, photochemistry reaction products, interstrand crosslinking products, mismatched bases, and other types of “damage” to the nucleic acid. Modified nucleotides can be caused by exposure of the DNA to radiation (e.g., UV), carcinogenic chemicals, crosslinking agents (e.g., formaldehyde), certain enzymes (e.g., nickases, glycosylases, exonucleases, methylases, other nucleases, glucosyltransferases, etc.), viruses, toxins and other chemicals, thermal disruptions, and the like.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “oligonucleotide” and “polynucleotide” may be used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

The term “nucleic acid” and “polynucleotide” may be used interchangeably to refer to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

For example, a template polynucleotide chain may be any sample that is to be sequenced, and may be composed of DNA, RNA, or analogs thereof (e.g., peptide nucleic acids). The source of the template (or target) polynucleotide chain can be genomic DNA, messenger RNA, or other nucleic acids from native sources. In some cases, the template polynucleotide chain that is derived from such sources can be amplified prior to use. Any of a variety of known amplification techniques can be used including, but not limited to, polymerase chain reaction (PCR), rolling circle amplification (RCA), multiple displacement amplification (MDA), or random primer amplification (RPA). It is to be understood that amplification of the template polynucleotide chain prior to use is optional. As such, the template polynucleotide chain will not be amplified prior to use in some examples. Template/target polynucleotide chains can optionally be derived from synthetic libraries. Synthetic nucleic acids can have native DNA or RNA compositions or can be analogs thereof.

Biological samples from which the template polynucleotide chain can be derived include, for example, those from a mammal, such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such s Pneumocystis carinii Takifugu rubripes, yeast, Saccharamoyces cerevisiae or Schizosaccharomyces pombe; or a Plasmodium falciparum. Template polynucleotide chains 48 can also be derived from prokaryotes such as a bacterium, Escherichia coli, staphylococci or Mycoplasma pneumoniae; an archae; a virus such as Hepatitis C virus, ebola virus or human immunodeficiency virus; or a viroid. Template polynucleotide chains can be derived from a homogeneous culture or population of the above organisms or alternatively from a collection of several different organisms, for example, in a community or ecosystem.

Moreover, template polynucleotide chains may not be derived from natural sources, but rather can be synthesized using known techniques. For example, gene expression probes or genotyping probes can be synthesized and used in the examples set forth herein.

In some examples, template polynucleotide chains can be obtained as fragments of one or more larger nucleic acids. Fragmentation can be carried out using any of a variety of techniques known in the art including, for example, nebulization, sonication, chemical cleavage, enzymatic cleavage, or physical shearing. Fragmentation may also result from use of a particular amplification technique that produces amplicons by copying only a portion of a larger nucleic acid chain. For example, PCR amplification produces fragments having a size defined by the length of the nucleotide sequence on the original template that is between the locations where flanking primers hybridize during amplification. The length of the template polynucleotide chain may be in terms of the number of nucleotides or in terms of a metric length (e.g., nanometers).

A population of template/target polynucleotide chains, or amplicons thereof, can have an average strand length that is desired or appropriate for a particular sequencing device. For example, the average strand length can be less than about 100,000 nucleotides, about 50,000 nucleotides, about 10,000 nucleotides, about 5,000 nucleotides, about 1,000 nucleotides, about 500 nucleotides, about 100 nucleotides, or about 50 nucleotides. Alternatively or additionally, the average strand length can be greater than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. Alternatively or additionally, the average strand length can be greater than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. Alternatively or additionally, the average strand length can be greater than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The average strand length for a population of target polynucleotide chains, or amplicons thereof, can be in a range between a maximum and minimum value set forth above.

In some cases, a population of template/target polynucleotide chains can be produced under conditions or otherwise configured to have a maximum length for its members. For example, the maximum length for the members can be less than about 100,000 nucleotides, about 50,000 nucleotides, about 10,000 nucleotides, about 5,000 nucleotides, about 1,000 nucleotides, about 500 nucleotides, about 100 nucleotides or about 50 nucleotides. For example, the maximum length for the members can be less than about 100,000 kilo nucleotides, about 50,000 kilo nucleotides, about 10,000 kilo nucleotides, about 5,000 kilo nucleotides, about 1,000 kilo nucleotides, about 500 kilo nucleotides, about 100 kilo nucleotides or about 50 kilo nucleotides. For example, the maximum length for the members can be less than about 100,000 mega nucleotides, about 50,000 mega nucleotides, about 10,000 mega nucleotides, about 5,000 mega nucleotides, about 1,000 mega nucleotides, about 500 mega nucleotides, about 100 mega nucleotides or about 50 mega nucleotides. Alternatively or additionally, a population of template polynucleotide chains, or amplicons thereof, can be produced under conditions or otherwise configured to have a minimum length for its members. For example, the minimum length for the members can be more than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. For example, the minimum length for the members can be more than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. For example, the minimum length for the members can be more than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The maximum and minimum strand length for template polynucleotide chains in a population can be in a range between a maximum and minimum value set forth above.

As used herein, the term “read” or “sequence read” refers to a raw sequence that comes off a sequencing machine. A read may consist of multiple segments. For sequencing data, reads are indexed by the order in which they are sequenced. In DNA sequencing, a read is an inferred sequence of base pairs (or base pair probabilities) corresponding to all or part of a single DNA fragment. A typical sequencing experiment involves fragmentation of the genome into millions of molecules, which are size-selected and ligated to adapters. The set of fragments is referred to as a sequencing library, which is sequenced to produce a set of reads.

As used herein, the term “links file” refers to information about overlapping read sequences, including the length of the overlap, the number of mutations in the overlapping region, and the relative orientation of the overlap (forward or reverse complement). The relative orientation is also referred to as a “polarity” and is allowed to take on values of 1 (same orientation), −1 (the read overlap is a reverse complement for one of the reads), or 0 (the orientation is unknown).

As used herein, the term “guides” refers to segments of a reference genome.

As used herein, the term “MUT reads” refers to reads with an artificial mutation, introduced to allow the reads from a single template molecule to be grouped together

As used herein, the term “INDEL” refers to insertions/deletions.

As used herein, the term “SNP” refers to single nucleotide polymorphisms.

As used herein, the term “template” refers to a DNA/RNA sequence part of which is sequenced on a sequencing machine or assembled from raw sequences.

As used herein, the term “segment” refers to a contiguous sequence or subsequence.

As used herein, the term “linear alignment” refers to an alignment of a read to a single reference sequence that may include insertions, deletions, skips and clipping, but may not include direction changes (i.e., one portion of the alignment on the forward strand and another portion of alignment on reverse strand). A linear alignment can be represented in a single SAM record.

As used herein, the term “read alignment” refers to a linear alignment or a chimeric alignment that is the complete representation of the alignment of the read.

As used herein, the term “multiple mapping” refers to situations where the correct placement of a read may be ambiguous, e.g., due to repeats. In this case, there may be multiple read alignments for the same read. One of these alignments is considered primary. All the other alignments have the secondary alignment flag set in the SAM records that represent them. Usually, the SAM records have the same QNAME and the same values for 0x40 and 0x80 flags. Typically, the alignment designated primary is the best alignment, but the decision on primary alignment may vary.

As used herein, the term “tracing” refers to constructing, from a k-mer graph, a sequence of nodes that joins a sequence of edges. For example, tracing may refer to a random walk on a k-mer graph that samples potential k-mer sequences. In a k-mer graph, a node may represent a k-mer, and edges may represent co-occurrences between a k-mer and an adjacent k-mer, and optionally edge direction may represent the ordering of k-mers. All nodes and edges may or may not be distinct. Edges may or may not be directed. Tracing may optionally begin by identifying nodes that have no nodes leading into them, such that those nodes are the beginning of potential paths to be traced.

As used herein, the term “BAM file” refers to the binary version of a SAM file. A SAM file (.sam) is a tab-delimited text file that contains sequence alignment data. These formats are described further online. BAM, rather than SAM, is the recommended format for IGV. Starting with IGV 2.0.11, IUPAC ambiguity codes in BAM files are supported. Binary Alignment Map (BAM) is the comprehensive raw data of genome sequencing; it consists of the lossless, compressed binary representation of the Sequence Alignment Map-files. BAM is the compressed binary representation of SAM (Sequence Alignment Map), a compact and index-able representation of nucleotide sequence alignments. The goal of indexing is to retrieve alignments that overlap a specific location quickly without having to go through all of them. Before indexing, BAM must be sorted by reference ID and then leftmost coordinate. BAM is in compressed BGZF format.

The structure of BAM files may include a header section and an alignment section:

[1] Header—The sample name, sample length, and alignment method are all included in this section. The alignments section contains alignments that are linked to specific information in the header section.

[2] Alignments—The read name, read sequence, read quality, alignment information, and custom tags are all included in this file. The chromosome, start coordinate, alignment quality, and match descriptor string are all included in the read name. Alignment Section includes the following: Read Group (RG), Barcode Tag (BC), Single-end alignment quality (SM), Paired-end alignment quality (AS), Edit distance tag (NM), and Amplicon name tag (XN)

The BAM format uses a 0-based coordinate system, whereas the SAM format uses a 1-based coordinate system. The BAM format can represent values in the range [—2³¹, 2³²]

### Methods of the Disclosure

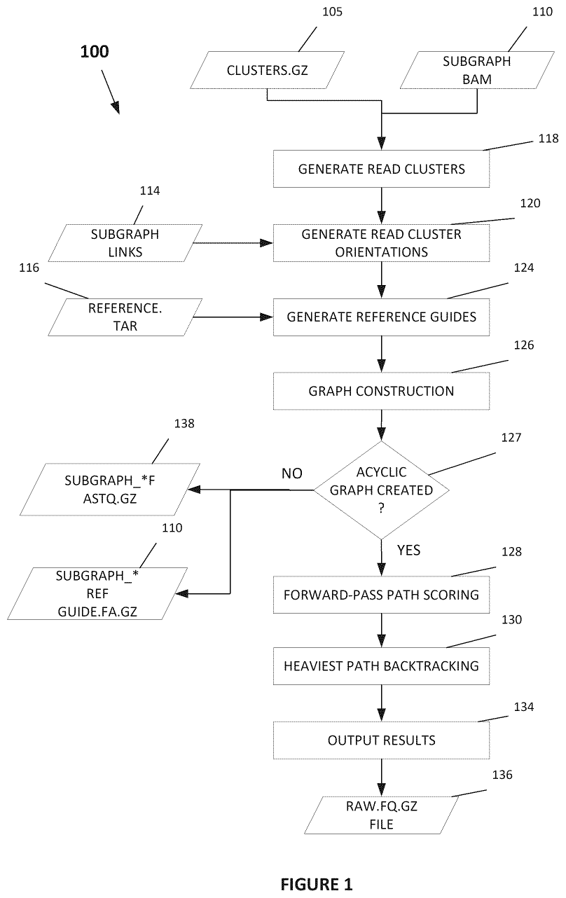

FIG. 1 shows an overview of a guided assembly process 100 according to one embodiment. The process takes inputs from a clusters.gz file 105 and a subgraph BAM file 110. The clusters.gz file is a compressed file storing the reads and read pairs that are associated with each cluster from the sequencing run. A line in this clusters.gz file may have a line per cluster and each line would have the identifier for each read pair associated with the cluster numbered sequentially from 1 to n. It should be noted that each line may identify all of the read pairs associated with a cluster, and thus may have numerous read pair identifiers for each cluster.

The subgraph BAM file is a coordinate-sorted BAM file of all reads that are referred to in a file of the sequence clusters from an NGS process and their alignment to a template or reference genome. In one embodiment, the entire NGS sequence run can be subsetted into a plurality of BAM files, each containing a fraction of the total reads from the sequencing run. This allows the overall k-mer assembly process of assembling millions of clusters by creating subsets of clusters and their related BAM files, each, for example, into hundreds of separate k-mer subgraphs. Subsetting the BAM file may facilitate the data processing requirements of managing the entire data set which can be a terabyte or more of data to manage.

In some embodiments, methods may split the overall sequencing problem of, for example, several millions of clusters and/or assemblies into, for example, fewer than a thousand subgraphs. In some embodiments, ten million assemblies may be split into any of: 10,000, 5,000, 1,000, 700, 500, 300, and 100 subgraphs. In some embodiments, five million assemblies may be split into any of 10,000, 5,000, 1,000, 700, 500, 300, and 100 subgraphs. In some embodiments, one million assemblies may be split into any of 10,000, 5,000, 1,000, 700, 500, 300, and 100 subgraphs. A typical size for an overall data set may comprise billions of read pairs, and may be 10 billion, 8 billion, 6 billion, and 4 billion read pairs. Such data sets may approach a terabyte in size and, therefore, would not fit into RAM on commodity hardware. Embodiments of the disclosure may advantageously subset the assembly size to fit into typical computer hardware or optimize the file of processing on FGPA hardware (as opposed to processing on a processor). In some embodiments, the input data will be in the same format as a typical BAM or SAM file. A typical input file may include a line per cluster, and each line may include an identifier. An identifier may be an integer identifier for each read pair numbered sequentially from 1 to an arbitrarily large number N.

In addition, overlapping similar read pairs can be determined by matching read pairs with one another. This allows the system to create links between the overlapping similar read pairs which can be stored in a subgraph links file 114. The subgraph link file stores links between a pair of read pairs. In some embodiments, these linked read pairs share sequence homology with one another. In some embodiments, the process of artificially mutating the original template sequence results in identifiable mutations in the pairs of read pairs which assists in determining the sequence homology and overlap between sets of read pairs. The links identify pairs of read pairs that are similar to each other in overlap and may also specify the orientation that each read pair overlaps with other read pairs. This allows the system to have knowledge of the nucleic acid orientation of each read pair as a read pair is matched with other similar read pairs. For example, if two read pairs are oriented in the same direction as compared to the template, the system may determine that they are positively oriented, and if they are oriented in opposite directions compared to the template the system may determine they are negatively oriented. Those orientations can be used later in the assembly process.

The process 100 then moves to a box 118 where read clusters are generated by pulling the sequences of all of the read pairs that are involved in a single cluster together in memory so that they are sequentially accessible to the system. Thus, this step gathers the sequences for a given cluster in memory for later processing by the system. In an embodiment of the disclosure, the process may use standard BAM files to generate read clusters. For example, read clusters may be generated by inputting a cluster file to the assembly system, wherein the cluster file contains one cluster per line. Each line of the cluster file may include a listing the IDs of the read pairs composing that cluster. This cluster file may be parsed to create a map associating each read ID to its cluster ID, where the ID of a given cluster may be its line number. Each line may be parsed independently, and each read ID composing the line may be stored in a cluster map as a key. An example value of the key may be the line number of the associated cluster.

After parsing, a vector of reads may be created for each cluster. The BAM file may be processed using the read-to-cluster ID map. In some embodiments, the map may be used for each record to find the cluster ID associated with the read ID. The read may be pushed into the vector of reads corresponding to that cluster. The disclosed methods may output a vector associating each cluster ID to the vector of reads contained in the cluster.

The process 100 then moves to box 120 wherein the subgraph links information to gather orientation information for each of the read pairs from each cluster. This information can be used to determine if a given read pair is in the correct orientation with its linked read pairs. If orientation information is given to the system, the system may use that orientation information, such as orientation information 114 to help build the k-mer graph for a particular sequence. In some embodiments, generating cluster read orientations proceeds by parsing the binary subgraph links file in a single sequential pass, during which a “union-find” data structure is populated. Once this data structure is fully populated, the union-find data structure may record three pieces of information: a) an assignment of each read-pair to a connected component in the graph, b) the read ID of a representative read for each connected component, which may be the smallest (absolute value) read ID in the component, and c) the orientation of each read in a connected component relative to the representative read.

In order to build the union-find data structure, a representative vector may be initialized with length equal to the maximum read ID. Each element of the representative vector may record two pieces of information: the representative read ID of the connected component that the read currently belongs to, and the orientation relative to the representative (i.e. the polarity). The representative ID for entry i may be initialized to i, and the polarity may be initialized to 1. In the alternative, where the set of read IDs is sparse the use of an unordered map/hash table may lead to a more memory-efficient implementation. These functions may output a data structure that enables the relative orientations of reads in a cluster to be queried.

If the orientation of the reads is negatively oriented, the system can reorient the sequence data for the read pair so that all the linked read pairs associated with the cluster can be oriented in the same direction as the single target sequence being assembled.

In the next step, the reference genome information from a reference file 116 is used as input into the box 124 to generate reference guides. This allows a determination of where along the reference genome the read pairs align. This data can be used to estimate which read pairs may plausibly align with particular sections of the reference genome or template. From this estimation, some, or no, estimated alignments of the linked read pairs may be determined from each cluster with the reference genome.

In generating a reference guide for a given cluster, the assembly system may iterate through associated reads and clusters, and infer the proper reference guide according to the mapping positions of the reads. An input to generate the reference guide may be a BAM file containing reference-sorted mapping of MUT reads associated to a cluster. This step 124 may iterate through all the paired reads from a cluster, and use their mapping positions to extract the reference guides of the cluster. For a given read, the reference guide may be first initialized according to the mapped beginning and end positions of that read. Since the input BAM file may be coordinate-sorted, while processing the following reads, the previous reference guide may be extended to account for the current read's mapping coordinates, if two reads are within a given range. If the two reads are not within a given range, a new reference guide may be initialized for the current read, and the same process may be applied to all reads.

In some cases, thresholds may be set to exclude reference guides that may be too short, or an insufficient number of reads may cover that. In some embodiments, a lower threshold may be a reference guide with at least two reads and/or read pairs mapping to the reference guide, where at least one of the two reads must have a strong alignment, e.g. mapQ >=20. In some embodiments, the lower threshold for a reference guide may also require that the reference guide has a length of at least 250 bp. In some embodiments, a lower threshold may be set to exclude reference guides that are below 500, 400, 300, 200, 100 or fewer nucleotides in length. Such guides may still be extracted by the aforementioned process, but not be output if they are below the set threshold values.

If orientation information was provided for the reference guide, the orientation of both the reads and the reference guides may also be updated accordingly during this step 124. Using the data structure populated during the previous step, each read of each read pair may be compared and oriented relative to the representative of the cluster and relative to the orientation indicated in the BAM file. For a given read belonging to a given pair, the following formula may be used, where 1 indicates a forward orientation, and −1 indicates a reverse orientation:
orientation [read]=relativeOrientation(pair, representative)*(isFirstMate (read)? 1:−1)*BAMSign(read) where relativeOrientation(pair, representative) returns 1 if both pairs have the same orientation, −1 if they don't, and 0 if their relative orientation is unknown.

This process may be useful for unmapped reads, for which some embodiments of the disclosure may be able to compute orientation relative to the representative of the cluster, and ensure the reads may be sent to the next steps of the pipeline in the right orientation. A similar procedure may be applied to reference guides, for which orientation may be computed based on the orientation of each individual mapped read, and for which a majority vote may be performed. If most reads align to a given guide in reverse orientation, the guide may be reverse-complemented, and aligned reads may be reverse-complemented accordingly.

An output of this process may be a vector containing all the reference guides associated to the cluster that passed the quality thresholds, as well as a vector of pile-ups, recording read events information for each position of each reference guide. A boolean value may also be returned to indicate whether the cluster should be sent to the k-mer assembler. In cases where a reference guide may be too long, or where a cluster may contain too many reads, this boolean may be set to false, and the cluster may not be sent to the k-mer assembly system. This step 124 may also return the ID, chromosome, and start and end positions of the most supported reference guide for the cluster.

This reference guide data may be used for the later assembly graph process, even if not all of the linked read pairs were able to be matched to a particular location along the reference genome. For example, in the process of generating a reference guide, the assembly system may align a particular read pair from a cluster to a location A on the reference genome. The system may then align a linked read pair from the same cluster to a location B on the same reference genome. If location A and location B are within a short distance of one another then the likelihood that the alignment was correct is greater because two reads which are linked to one another should be near neighbors on the reference genome. However, if location A and location B are not near each other, the likelihood that both linked read pairs were properly aligned is lower. For example, if location A and location B are within 5000, 4000, 3000, 2000, 1000, 500 or fewer bases then the linked read pairs may be considered to be properly aligned with the reference genome. This process can then be repeated for every read pair associated with a particular cluster so that each read pair can be aligned with the reference genome and a measure of how correlated the read pairs from the cluster are with the reference genome can be determined. In general, the more read pairs which are found to align with the reference genome, the more confident the system can be that the alignment is correct. If the confidence that a reference guide is found with sufficient evidence, then the particular guide can be added into the assembly process. This process is repeated for every linked set of read pairs for every cluster and the guides which are found to contain sufficient alignments with a number of read pairs from a cluster are all added to the assembly process. In one embodiment, the map/align process may be used to filter sequences based on a confidence score for a given alignment or location. In some embodiments, any method or process known in the art may be used to align read pairs with the reference genomes.

The reference guides and their aligned, oriented, linked read pairs are then input into the graph process at box 126. The graph construction process may be a k-mer based construction process, such as outlined in U.S. Pat. Nos. 14,775,252 and 17,165,828, both herein incorporated by reference in its entirety. An input for this graph process step may be the set of reads and the set of reference guides associated to a cluster. Contiguous sequences of sufficient quality bases may be extracted from those reference guides and reads, to form subsequences which are then used to create k-mers of a specific length. It should be realized that this length may be adjusted, and the graph construction process may be restarted using multiple different lengths, if necessary, to produce an effective k-mer graph during the graph assembly process.

The k-mers generated from the reference guides may be tagged as such for later differentiation from k-mers generated by covering reads. In particular embodiments, the k-mers derived from reference guides (e.g., the k-mers that do not appear in any read), may not contribute to the edge weights of the graph. Furthermore, the uniqueness of k-mers may be tracked in the domain of each subsequence. For example, this may mean that a particular k-mer may only be considered as non-unique if it occurs multiple times in a single subsequence. According to some embodiments, a k-mer occurring multiple times across multiple subsequences, but a single time across each individual subsequence, would not be considered non-unique.

In some embodiments, graph construction may begin with k-mers from the reference guides being used to construct the backbone of a k-mer graph, which may in turn be used to guide the assembly of the k-mer graph. Each reference guide may be processed independently, and through a process of threading, k-mers derived from the reference guides may be sequentially added to the k-mer graph. Each k-mer that does not already exist in the graph thus induces a new vertex, with zero or more edges connecting to shared k−1 mers. As a result, if a new fork is created, or if an existing path is extended, then new vertices and edges may be added, with initial edge counts being zero, since reference guides may be not allowed to contribute to edge weights. At the end of this step after all the k-mers from the reference guides have been added to the graph, all edges of the graph have weight 0.

Moreover, since multiple reference guides can be provided, a graph obtained after such a first step may have multiple sources and multiple sinks. After processing all reference guides, through the same process, the k-mers derived from covering reads may be added to the graph, and edge counts in the graph may be incremented as reads thread through existing paths. If a new fork is created, or if an existing path is extended, then new vertices may be added, but the initial edge count may be set to 1.

If a cycle is created, or if the graph is of too low a complexity as determined by the % of k-mers which may be non-unique, then construction may be aborted to retry with a different k-mer length, see, for example, step 127. After a fork, paths can typically only rejoin on nodes corresponding to k-mers which may be in the unique set, meaning only unique k-mers can branch. It should be realized that a unique k-mer might not necessarily be unique across the set of all k-mers but may be unique in the context of a single subsequence, and then joined to the set of all unique k-mers. Moreover, reference-guide k-mers may not be required to be unique for graph construction to be successful at a given k-mer length. The output may be a successful graph having the lowest k-mer length, which, by construction, does contain any cyclic k-mers.

As mentioned above, the assembly system may build test graphs using k-mers of different sizes to determine a graph which has no cycles in it. In some embodiments, the smallest k-mer which creates a useful graph may be selected. In some embodiments, the k-mers may range in size from 10 to 100 nucleotides in length, although fewer or greater numbers of nucleotides per k-mer are contemplated. The k-mer graph contains nodes and edges. The nodes are oligonucleotide sequences and the edges imply that two oligos are identical to each other, except, for example, for the first base of one oligo and the last base of the next oligo. Accordingly, such an edge implies that a k-mer at a particular node may be extended by a single base to produce another k-mer that is another node in the k-mer graph.

An edge in a k-mer graph is an indication of the support for a particular k+1 nucleotide from the oligonucleotide at the related node in the graph. One feature provided during the disclosed assembly and traversal through the graph is the amount of support a particular edge has in the sequence and read data that made up the graph and the assembly problem. In one embodiment, the count of reads which have an exact match to the sequence implied by the edge may be used to calculate the weight of that edge. By way of example, if there is one read which has a particular edge k-mer, then the weight of that edge may be set as 1. If there are five reads which have the particular edge k-mer then the weight of that edge may be set to 5, and so on.

In order to determine a reconstruction of the target reference genome the system may choose a single forward pass path through the k-mer graph at box 128. In some embodiments, methods of the disclosure may employ heaviest path extraction. In some embodiments, a single heaviest path may be computed, but it is expressly considered that multiple paths could be extracted (especially in the case of multiple reference guides). An example function for heaviest path extraction may operate as follows by first performing a forward-pass path scoring on the k-mer graph. The weight of each possible path may be defined as the sum of the path's edges weights, which may be used to determine the heaviest possible forward path. This step may apply a dynamic-programming forward pass, resulting in each node knowing the weight of the best (partial) path terminating at that node and the identity of the edge leading into the node (if any) that is included in the best path to the node. The path extraction is not necessarily constrained to start and stop on reference guides beginning and end nodes, and the extracted path can thus start and stop at any node. A single heaviest path may be computed, but in the case of multiple reference guides, multiple paths could be extracted, e.g., by iteratively finding and removing the heaviest path from the graph on each iteration. At the end of the step, the node with the heaviest path ending at the node is known; this is the “heaviest-path end node”. The disclosed function may output the node of the graph corresponding to the last node of the heaviest path.

In some embodiments, such a function may assign a quality score to the long template bases, based on local maximum edge weight, and/or identify and mark bases supported only by the reference guides. The disclosed function may output an assembled long template, with a quality score assigned to each base.

An input for these steps 128 or 130 may be the k-mer graph generated during a previous step, which would not contain a cycle if graph construction was successful. The path extraction may be directly performed on the k-mer graph. The path is not constrained to be strictly head to tail and can thus start and stop at any node. The weight of each path may be defined as the sum of the path's edge weight. In some embodiments, all graph nodes may be looped over, and nodes with no incoming edge may be added to a queue. For each node, a best weight and best length values may be recorded, as well as a backtrack pointer, indicating the previous node of the best path associated to that node, which may be used in the following backtracking step.

After initializing the queue, nodes pushed into it may be populated one by one. For each node, if its best weight value is strictly greater than the current global best weight that has been found (initialized to 0), the latter value may be updated according to that of the node, and a pointer to that node may be recorded as the best tail. All outgoing edges of the current node, which may be referred to as the source, may then be processed. Each node that can be reached following one of those edges may be called a target. If the sum of the source's best weight and the followed edge's weight is strictly greater than the target's best weight, the target's best weight and best length values may be set accordingly, and the target's backtrack pointer is set to the source. Since reference might not be allowed to contribute to edge weights, such embodiments may ensure that the head and tail of the path would not contain reference-only nodes, and, in turn, the resulting long template would not contain heading/tailing bases originating solely from reference guides.

If all incoming edges leading to a target have been visited, there would be no additional ways of travelling through the graph to arrive at that target. In the same manner as for the queue initialization, the target may be added to the queue, so that all nodes of the graph can be processed following the same process. Making sure all incoming edges may be visited before adding a node to the queue would ensure that every node is only processed once. The processing of single nodes selected from the queue continues until a queue is empty, at which point embodiments of the disclosure will have iterated through all the nodes of the graph. An output may be the graph node marked as the best tail after all nodes have been processed.

The process 100 then can determine the heaviest path extraction and backtracking at a box 130. This process can start at the last node in the path which had the highest total sum of edge weight scores. Beginning with the last node in that path, the system moves backwards though the path and calculates the k-mers to determine the sequence associated with each node as the path is traversed. If a node is reached which has multiple possible paths into it, the system may choose the path with the highest edge weight score to traverse as it moves backwards through the path. The system continues iterating this process backwards though the path, moving through the path by choosing the highest weighted edges for each node in the path until the first node is reached. Once the final backtracking path through the k-mer graph is determined, the system can construct the nucleotide sequence of the nodes making up the path since each node differs from the prior node by a single nucleotides in the graph.

As mentioned above, multiple forward paths through the k-mer graph could be extracted, and the backtracking process could thus be applied to each of those paths. An input for this backtracking step may be a pointer to the graph node marked as the “best tail”. A “best tail” may be any tail that a forward pass extraction process identifies as a beneficial tail end of a path through the k-mer graph. In some embodiments, a best tail may be a locally optimal tail for a best path identified during a forward path extraction. The steps, including 130, perform backtracking of the heaviest path to generate a long template. Backtracking may be performed from the best tail until the process reaches a node for which the backtrack pointer is not set, indicating the head of the heaviest path was reached.

For each node traversed this way the weight of the edge leading from the backtrack pointer to the current node may be added to the end of an edge weights list. This list may be constrained to contain at most K+1 values, where K is the k-mer size of the graph, in order to assign quality values to the assembled long template bases, based on a local maximum edge weight. For each node, its last base may be prepended to the result long template and may be assigned as a quality value a function of the maximum value present in the edge weights list. Given how the list is constructed, this embodiment amounts to each base being assigned a quality value depending on the number of reads the base was supported. The first element of the list may then be removed, if the list contains the maximum allowed number of values, and the backtracking process continues with the node that is pointed to by the backtrack component points. To be more precise, each quality value may be computed using the following formula, where maxEdgeWeight represents the maximum value in the list at a given time, and where 33 is an offset used for ASCII encoding: quality Value=min (maxEdgeWeight >0? 20+2*(maxEdgeWeight−1): 0,40)+33. This strategy also provides for identification and marking of bases that might be only supported by reference guides, assigning them a quality value of 0.

When reaching the head node (e.g., the node for which the backtrack pointer is not set), each base may be prepended to the result long template one by one and may be assigned as a quality value the maximum value present in the edge weights list. After treating each base in the same fashion as previously, the first element of the edge weight list may be removed to maintain the local maximum edge weight logic for quality value assignment. An output may be an assembled long template, with a quality value assigned to each base.

One aspect of the disclosure is directed to methods and applications for k-mer graph construction. In some embodiments, the k-mer assembly method may differ from a typical k-mer assembly process. For example, in some embodiments, the guide sequences would be copied from within the reference genome and not counted when assigning edge weights. Instead, edge weights according to some embodiments may be due to the actual sequence reads that go into the assembly process.

In the trivial case a graph may be constructed where one k-mer is followed by another k-mer and can be extended by one base to the next along the next edge, extended by another base along the next edge, all the way onto the end of the sequence. A graph may be constructed with all of the k-mers that are in the reads and the guides that were input that graph by generating a straight line straight line of k-mers. However, this assembly method can be complicated in practice for a few reasons. Realistic graphs are generally more complicated with splits where the path may branch in two different directions that do not come back to each other, and bubbles where the path might bifurcate, for a while, in a long slightly different sequences and then come back.

To address these concerns, some embodiments may proceed by choosing a single path through the graph wherein the sum of the edge weights on that path is higher or as high as any other possible path through the graph. Edge weights may be used advantageously to produce sequences from k-mer graphs.

Some embodiments of the disclosure may use a dynamic programming technique to assist in assembly. Dynamic programming generally refers to computer programming techniques where a problem is first broken down into sub-problems, the results are saved, and then the sub-problems are optimized to find the overall solution. Using a dynamic programming technique, some embodiments may start by first identifying nodes that have no nodes leading into them, such that those nodes are the beginnings of potential paths. Then assembly may proceed by tracing from each of those nodes and to build up a sum of edge weights until the nodes have two possible ways of getting there, or two possible ways of going out. Some embodiments have different bookkeeping methods to sum edge weights. Embodiments may proceed by finding a node path's best possible score for a given node that is traced and whether the trace comes in from the top or the bottom or bottom of the node (i.e., referring to the two options for going into the node).

In some embodiments, methods according to the disclosure may determine the best partial path for some subset of nodes. In some embodiments, methods may employ edge weight propagation or forward pass path scoring techniques to choose the best route. Accordingly, each node may be associated with the best possible weight of any path that reaches the node. The node may then provide information about which immediately preceding node provided the best Partial path up to that point. Eventually, there will be some nodes that do not have anywhere to continue. Then a single best score may be calculated and selected from all those terminal nodes. For example, a single best score may be the heaviest path from a forward pass scoring.

In some embodiments, methods may use backtracking to produce a complete path. Each terminal node, because these nodes have no way to walk back to a different preceding node, would only create a single sequence from the k-mer of the node itself. Accordingly, if a node has only one way to get to it (i.e., one edge into it), then disclosed methods may follow an edge backward to a preceding node and then operate on that node and try to keep going backward. However, if an initial node (non-terminal node) has more than one option to get to it, then each option has edge weights calculated during a first forward pass and an associated best score to that node. Accordingly, some embodiments may trace the best edge backward and may recurse or iterate on that process of following the best edge backward. Embodiments may proceed until reaching a beginning node of that path, which may be one node that has no nodes coming into it.

After recursion, some embodiments will have gathered all relevant nodes and associated scores. Such embodiments may extract a sequence from all nodes from a unique path within the k-mer graph. In some embodiments, confidence scores may be assigned to individual edge weights and to sums of edge weights for different traces. A large edge weight score may represent relatively high confidence in a potential sequence, while low edge weights may be less preferred and provide relatively lower confidence in a potential sequence. In some embodiments, alternative path information may be retained for some or all of the sequencing process and may assist in finding a global optimum. Methods according to the disclosure may provide information comprising a sequence and confidence scores that may be output as an assembled sequence. This information may be referred to as a contig, which is a well-known term in the assembly literature. Contigs may be output as fastQ comprising sequence data and confidence scores.

That assembled sequence or contig may then be used as part of the results that are output at a box 134. That sequence may then be then output as a compressed fastq file 136.

It should be realized that in some cases, the graph construction process which occurs at box 126 may fail to create a useful k-mer graph. For example, the graph may only contain cycles where each path through the graph cycles back upon itself so there is no linear path through the graph. In that case, the system may output a set of subgraph files which contain data relating to the oriented reads for each cluster in a fastq file 138 or the oriented reference guide from each cluster 140 from the failed graph construction. It should be realized that if the graph construction process fails at box 126, the process 100 may move directly to output the files 138, 140 and not continue along with any of the further processes at boxes 128, 130, 134, or 136.

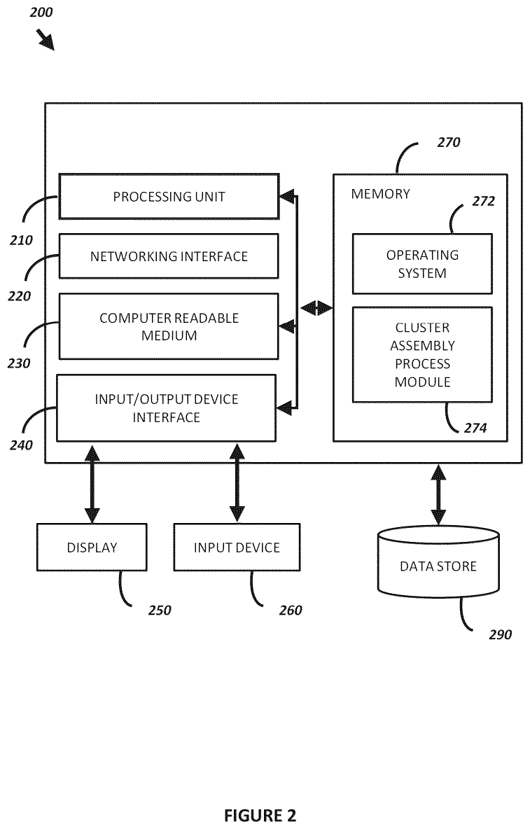

Embodiments of the present disclosure may also include a system for analyzing and assembling sequences of polynucleotides. FIG. 2 is a block diagram of an exemplary computing system 200 that may be used in connection with an illustrative sequencing system. The computing system 200 may be configured to determine a DNA sequence by using the sequencing and assembly methods disclosed herein. The general architecture of the computing system 200 depicted in FIG. 2 includes an arrangement of computer hardware and software components. The computing system 200 may include many more (or fewer) elements than those shown in FIG. 2. It is not necessary, however, that all of these generally conventional elements be shown in order to provide an enabling disclosure.

As illustrated, the computing system 200 includes a processing unit 210, a network interface 220, a computer readable medium drive 230, an input/output device interface 240, a display 250, and an input device 260, all of which may communicate with one another by way of a communication bus. The network interface 220 may provide connectivity to one or more networks or computing systems. The processing unit 210 may thus receive information and instructions from other computing systems or services via a network. The processing unit 210 may also communicate to and from memory 270 and further provide output information for an optional display 250 via the input/output device interface 240. The input/output device interface 240 may also accept input from the optional input device 260, such as a keyboard, mouse, digital pen, microphone, touch screen, gesture recognition system, voice recognition system, gamepad, accelerometer, gyroscope, or other input device.

The memory 270 may contain computer program instructions (grouped as modules or components in some embodiments) that the processing unit 210 executes in order to implement one or more embodiments. The memory 270 generally includes RAM, ROM and/or other persistent, auxiliary or non-transitory computer-readable media. The memory 270 may store an operating system 272 that provides computer program instructions for use by the processing unit 210 in the general administration and operation of the computing device 200. The memory 270 may further include computer program instructions and other information for implementing aspects of the present disclosure.

For example, in one embodiment, the memory 270 includes a cluster assembly process module 274 for analyzing and assembling sequences of polynucleotides. The cluster assembly process module 274 can perform the methods disclosed herein, including the process described with respect to FIG. 1. In addition, memory 270 may include or communicate with the data store 290 and/or one or more other data stores that store one or more inputs, one or more outputs, and/or one or more results (including intermediate results) of determining a DNA sequence and providing an assembly process according to the present disclosure. Such data may include, for example, unmutated sequence reads, mutated sequence reads, and reference guides.

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being Produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (for example, iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

One aspect of the disclosure is directed to a workflow module that may be integrated into existing workflows. In some embodiments, a workflow module may be a reference-guided assembly module and may be integrated into a NGS sequence analysis platform, for example the DRAGEN™ Bio-ID platform from Illumina. In some embodiments, a workflow module may use clustering information to extract aligned MUT reads and reference sequence segments, called reference guides, for each cluster. Each assembly cluster's set of reads and reference guides may be passed to an assembly system, to build a k-mer graph of all the sequences. If orientation information is provided, the assembly system may use the information to infer the proper orientation of the reads and reference guides. In practice, multiple graphs can be built with the assembly system, with different k-mer sizes for each graph. If, in some embodiments, some k-mer sizes do not produce satisfying graphs (e.g., graphs containing cycles or too many non-unique k-mers), these unsatisfactory graphs may be discarded. In some embodiments, once a satisfactory graph is built for some k-mer size, a heaviest path extraction procedure is applied to assemble a long template for the associated cluster, which is output to the results file. In some embodiments, if path extraction fails to produce a long template, the reads and reference guides associated with the clusters may be output to two different files (one for the reads, one for the reference guides), which are then optionally passed to an alternative assembly method. In some embodiments, if all k-mer sizes fail to yield a graph, the reads and reference guides associated with the clusters may be output to two different files (one for the reads, one for the reference guides), which are then optionally passed to an alternative assembly method. In some embodiments, the pathway for processing unsatisfactory graphs is deprecated.

In some embodiments, the k-mer graph assembly system may use clustering information to extract aligned MUT reads and reference guides of each individual assembly cluster. In some embodiments, the individual assembly clusters are determined prior to the main assembly process. From those aligned MUT reads and reference guides, a k-mer graph may be built. In some embodiments, if this resulting graph contains a cycle, or too many non-unique k-mers, construction can be aborted, and a larger k-mer size may be initiated and tested. In some embodiments, after building a satisfactory graph, a heaviest path extraction procedure may be applied to assemble a long template for the original cluster. In some embodiments, the same procedure may be applied independently to each assembly cluster. If path extraction fails, or if all k-mer sizes produce an unsatisfying graph, the reads and reference guides may be written to independent files, which may be passed to a fallback process.

In some embodiments, the assembler system or method may generate a compressed file, such as a gzipped fastq file containing long templates, and numbered according to the input subgraph ID, as an output. In some embodiments, a method may generate a csv file with timing details for steps in the assembly procedure, numbered according to the input subgraph ID, as an output. In some embodiments, a method may generate gzipped fastq files containing reads of the clusters that could not be assembled using the assembly system, to be passed to a fallback process as an output. In some embodiments, a method may generate compressed fasta files containing reference guides of the clusters that could not be assembled with the assembly process, to be passed to the fallback process as an output.

Embodiments can be part of a computer system according to some embodiments of the present disclosure. In some embodiments, the system may include a machine-readable memory, a processor and be configured to execute machine-readable instructions, which, when executed by the processor, cause the system to perform steps described herein. The processor may include a k-mer module which includes instructions that are configured to execute, for example, a method of assembling polynucleotides from k-mer fragments.

In some examples, the k-mer module can be implemented or performed by a programmed computer, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. The processor can be a microprocessor, but in the alternative, the processor can be a controller, microcontroller, or state machine, combinations of the same, or the like. A processor or group of processors for performing the methods described herein may be of various types including programmable devices (e.g., CPLDs and FPGAs) and non-programmable devices such as gate array ASICs or general-purpose microprocessors.

A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory. In some examples, a hardware platform for providing a computational environment may be used. The hardware platform may comprise a processor (e.g., CPU) and a memory such as random access memory (RAM). In some embodiments, graphics processing units (GPUs) can be used. In some embodiments, hardware platforms for performing computational methods as described herein comprise one or more computer systems with one or more processors. In some embodiments, smaller computers are clustered together to yield a supercomputer network. The hardware platform may be specially constructed for the required purposes, or it may be a general-purpose computer (or a group of computers) selectively activated or reconfigured by a computer program and/or data structure stored in the computer. In some embodiments, a group of processors performs some or all of the described functionalities collaboratively (e.g., via a network or cloud computing) and/or in parallel.

Elements of the methods or processes described herein can be embodied in a software module executed by a processor. A software module can reside in RAM memory, flash memory, ROM memory, EPROM memory, EEPROM memory, registers, hard disk, a removable disk, a CD-ROM, or any other form of computer-readable storage medium. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

### EXAMPLES

### Pseudocode for an Embodiment of Heaviest Path Extraction

In some embodiments, methods may employ a heaviest path extraction method to select a k-mer sequence. The path extraction may be directly performed on the k-mer graph. The path is not constrained to be strictly head to tail, and can thus start and stop at any node. The weight of each path may be calculated as the sum of its edges' weight. In some embodiments, the weight of each path may be calculated as a linear or non-linear expression of the edge weights. An example of pseudocode according to the disclosure proceeds as follows:

```
activeNodeQueue = [ ]
for node in nodes:
  if hasNoInNodes (node) :
    activeNodeQueue.append (node)
    node->bestWeight = node->weight
  node->bestWeight = 0
  node->bestLength = 1
  node->backTrack = None
globalBestWeight = 0 globalBestTail = None
# visit each edge once
while activeNodeQueue:
  active = activeNodeQueue.pop ( )
  if not active->outEdges: # if this node is a tail
  if active->bestWeight > globalBestWeight:
    globalBestWeight = active->bestWeight
    globalBestTail = active
  else:
    for edge in active->outEdges: target = edge->target
    bestWeightThisEdge = edge->weight + active->bestWeight
    if bestWeightThisEdge > target->bestWeight:
      target->bestWeight = bestWeightThisEdge
      target->bestLength = active->bestLength + 1
      target->backTrack = active
    target->inEdges.remove (edge)
    if not target->inEdges: # all the ways of getting to
  the target are visited; it can be added to active queue
      activeNodeQueue.append (target)
return globalBestTail
```

### Pseudocode for Heaviest Path Backtracking

In some embodiments, methods may employ a heaviest path backtracking extraction method to select a k-mer sequence. The path extraction may be directly performed on the k-mer graph, optionally after performing a first heaviest path procedure. The path may start and stop at any node. The weight of each path may be calculated as the sum of its edges' weight. In some embodiments, the weight of each path may be calculated as a linear or non-linear expression of the edge weights. An example of pseudocode according to the disclosure proceeds as follows:

```
heaviestPathSequence = [ ] heaviestPathQualities = [ ]
activeEdgeWeights = [ ] curNode = globalBestTail prevNode = null
remainingBases = globalBestTail->bestLength + klen − 1 # number
of bases of the heaviest path sequence
# Traverse each node of the heaviest path while curNode:
if curNode->backtrack:
  edge = curNode->getEdgeFrom (curNode->backtrack) # get edge
from previous to current node
    activeEdgeWeights.append (edge->weight)
  heaviestPathSequence.prepend (curNode->bases [klen-1] ) #
prepend last base of
the current node to the heaviest path sequence
  maxEdgeWeight = getMax (activeEdgeWeights)
  qualityValue = min (maxEdgeWeight > 0 ? 20 + 2 *
(maxEdgeWeight − 1) : 0, 40) + 33
  heaviestPathQualities.prepend (qualityValue)
# remove first element if list contains the maximum allowed
number of values if size (activeEdgeWeights) > klen or
remainingBases <
size (activeEdgeWeigths) :
  activeEdgeWeights.pop front ( )
  prevNode = curNode
  curNode = curNode.backTrack
  remainingBases-
# Last visited node contains the remaining first bases of the
heaviest path sequence
while remainingBases >= 0:
  heaviestPathSequence.prepend (curNode-
  >bases [ remainingBases] ) maxEdgeWeight =
  getMax (activeEdgeWeights)
  qualityValue = min (maxEdgeWeight > 0 ? 20 + 2 *
(maxEdgeWeight − 1) : 0, 40) + 33
heaviestPathQualities.prepend (qualityValue)
activeEdgeWeights. pop_front ( ) # remove first element if list
contains the
maximum allowed number of values remainingBases--
return heaviestPathSequence, heaviestPathQualities
```

### Generating Cluster Read Orientations

In some embodiments, methods of the disclosure generate cluster read orientations; however, this step is not required and may be skipped, if for example, no link orientation was provided. If no orientation data is provided, this step may be skipped in the overall workflow, and any reads may be further added to the graph in the orientation that the reads appear in the input BAM file.

In some embodiments, generating cluster read orientations proceeds as follows. A binary subgraph links file may be parsed in a single sequential pass, during which a “union-find” data structure is populated. Once fully populated, the union-find data structure records three pieces of information:
[1] an assignment of each read-pair to a connected component in the graph
[2] the read ID of a representative read for each connected component, which may be the smallest (absolute value) read ID in the component
[3] the orientation of each read in a connected component relative to the representative read

In order to build the union-find data structure, a “representative vector” may be initialized with length equal to the maximum read ID. Each element of the representative vector records two pieces of information: the representative read ID of the connected component that the read currently belongs to, and the orientation relative to the representative (i.e. the polarity). The representative ID for entry i may be initialized to i, and the polarity may be initialized to 1.

In some embodiments, a function such as “find_representative( )” may be used to carry out a union-find construction procedure. find_representative( ) takes as input a query read ID and returns the representative ID of the connected component representative and orientation of the query read relative to the representative read. Such an example may proceed by:

```
function find representative ( ) :
  1. set i = query read ID
  2. while representative [i].read_id != i
a. set parent = representative [i].read_id
b. set representative [i].read_id =
representative [parent].read_id
c. set representative [i].polarity = representative [i].polarity
* representative [parent].polarity
d. set i = parent
Each link in the input links file associates two read IDs, i
and j, and records the orientation of their overlap
(link.polarity). The following procedure may be used to
process each link:
1. set r_i to find representative (i), i.e. r_i is the id of
  the current representative for i
2. set r_j to find representative (j), i.e. r_j is the id of
  the current representative for j
3. set representative [r_i].read id = min(r_i, r_j)
4. set representative [r_j].read id = min(r_i, r_j)
5. if r_i < r_j then
  a. set representative [r_j].polarity =
  representative [j].polarity * link.polarity
    1. if r_j < r_i then
  b. set representative [r_i].polarity =
  representative [i].polarity * link.polarity
```

Finally, find_representative( ) may be invoked on every valid read ID in order to update it to point directly to the component representative read ID instead of pointing indirectly via another read. This completes the “path compression” part of union-find. Note that in practice the set of read IDs can be sparse and in this case, the use of an unordered_map/hash table can lead to a more memory-efficient implementation.

The illustrative embodiments described in the detailed description, drawings, and claims are not meant to be limiting. The teachings herein can be applied in a multitude of different ways, including for example, as defined and covered by the claims. It should be apparent that the aspects herein may be embodied in a wide variety of forms and that any specific structure, function, or both being disclosed herein is merely representative. Based on the teachings herein one skilled in the art should appreciate that an aspect disclosed herein may be implemented independently of any other aspect and that two or more of these aspects may be combined in various ways. For example, a system or apparatus may be implemented or a method may be practiced by one of skill in the art using any reasonable number or combination of the aspects set forth herein. In addition, such a system or apparatus may be implemented or such a method may be practiced using other structure, functionality, or structure and functionality in addition to or other than one or more of the aspects set forth herein. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and made part of this disclosure. It is to be understood that the disclosed embodiments are not limited to the examples described below, as other embodiments may fall within disclosure and the claims.

1. A method of assembling the nucleotide sequence of a polynucleotide from read pairs, comprising:
providing a flowcell comprising clusters of read pairs, wherein each read pair contains two sequence reads, and the two sequence reads are in opposite orientations of one another;
mapping a plurality of the read pairs to a reference guide of the polynucleotide;
orienting sequence reads in each read pair to be in the same orientation as the reference guide of the polynucleotide;
generating, from the oriented sequence reads, at least one k-mer graph comprising nodes and edges, wherein each node of the at least one k-mer graph corresponds to the read pairs from a single cluster and each edge has a weight corresponding to the number of identical sequence reads in the cluster; and
assembling the nucleotide sequence of the polynucleotide by tracing a plurality of acyclic paths through the at least one k-mer graph.

2. The method of claim 1, wherein assembling the nucleotide sequence of the polynucleotide comprises determining a path through the at least one k-mer graph with the highest sum of the edge weights.

3. The method of claim 1, wherein assembling the nucleotide sequence of the polynucleotide comprises selecting an acyclic path with the highest sum as the path for assembling the nucleotide sequence.

4. The method of claim 2, wherein assembling the nucleotide sequence comprises selecting the final node in each of the acyclic paths through the at least one k-mer graph.

5. The method of claim 4, wherein assembling the nucleotide sequence comprises tracing a reverse path through the at least one k-mer graph beginning with a final node.

6. The method of claim 5, wherein tracing the reverse path comprises selecting a final node in an acyclic path where the sum of the edge weights along the reverse path is the highest value compared to other reverse paths.

7. The method of claim 1, wherein assembling the nucleotide sequence of the polynucleotide comprises a globally optimum path of edge weights for each acyclic path through the at least one k-mer graph.

8. The method of claim 1, wherein mapping the read pairs comprises providing a cluster file, wherein the cluster file comprises read sequences, and an assignment of the read sequences to an assembly cluster.

9. The method of claim 8, wherein the cluster file comprises greater than 1 million read sequences.

10. The method of claim 1, wherein generating the at least one k-mer graph comprises generating a plurality of k-mer graphs.

11. The method of claim 10, wherein generating the at least one k-mer graph comprises generating greater than 100 k-mer graphs.

12. The method of claim 1, where the paired end length of the read pairs ranges from 25 bp to 10,000 bp.

13. The method of claim 1, wherein assembling the nucleotide sequence of the polynucleotide comprises tracing a path through an acyclic k-mer graph of the at least one k-mer graph.

14. A system for analyzing sequences of polynucleotides, comprising:
a machine-readable memory; and
a processor configured to execute machine-readable instructions, which, when executed by the processor, cause the system to perform steps according to claim 1.

15. A non-transitory computer-readable medium storing a polynucleotide sequence analyzing program including instructions that, when executed by a processor, causes a polynucleotide sequencing apparatus, to perform steps according to claim 1.
